(19) United States
(12) Patent Application Publication
(10) Pub. No.: US 2003/0195542 A1
(43) Pub. Date: Oct. 16, 2003

(54) Apparatus for making tattoo

(76) Inventor: Sang Ho Lee, Seoul (KR)

Correspondence Address: VENABLE, BAETJER, HOWARD AND CIVILETTI, LLP, P.O. BOX 34385, WASHINGTON, DC, 20043-9998, US

(21) Appl. No.: 10/410,407

(22) Filed: Apr. 10, 2003

(30) Foreign Application Priority Data
Apr. 10, 2002 (KR) 2002-0019409

(51) Int. Cl. A61B 17/34

(52) U.S. Cl. 606/186

(57) ABSTRACT

Provided is an apparatus for making tattoo that can reduce the violation of the apparatus and improve the tattoo making efficiency by forming a needle to perform a rectilinear reciprocation motion and go in and out of the case of the apparatus based on the rotation of the rotatory gear and longitudinal gear which are formed in a structure of helical bevel gear. The apparatus includes: a case for forming the outer side of the tattoo making apparatus to have a space inside; a driving motor inside the upper part of the case; a rotatory gear connected to a driving shaft of the driving motor; a longitudinal gear tooth-engaged with the rotatory gear; an eccentric shaft for converting rotatory motion to up-down rectilinear motion; a rotatory bar for performing an up-down rectilinear reciprocation motion based on the rotation of the eccentric shaft; a rectilinear bar for performing an up-down reciprocation motion based on the rotation of the rotatory bar; a rectilinear bar anti-rolling unit for preventing the rectilinear bar from rolling right and left; a needle for making a scar on the skin of a body; a connection unit for connecting the needle to another end of the rectilinear bar; and a grip provided with an exit for guiding the needle in the lower part of the case.

### FIELD OF THE INVENTION

The present invention relates to an apparatus for making tattoo; and, more particularly, to an apparatus for making tattoo that makes a scar on the skin of a body and inserts pigment into the scarred part of skin to express a character or figure by using a needle that performs a rectilinear reciprocation motion based on a motor that rotates at a high speed.

### DESCRIPTION OF RELATED ART

Generally, tattoo means a character or figure that is expressed on the skin of a body by making a scar on the skin and inserting pigment to the scarred skin, deliberately. Tattoo is originated from the marks Polynesian people used to make on their body.

In the initial days, tattoo is made by picking or piecing the skin with little tools made of an animal's bone to make a scar and inserting pigment into the scarred skin to leave an indelible mark on the skin. Today, various tattoo apparatuses with a needle performing a rectilinear reciprocation motion with help of a motor are developed and used. Following is the description on conventional tattoo apparatuses.

FIG. 1A is a cross-sectional view showing a conventional tattoo apparatus, and FIG. 1B is a cross-sectional view describing the operation of the tattoo apparatus shown in FIG. 1A. As described in FIGS. 1A and 1B, the conventional tattoo apparatus 10 includes: upper and lower part cases 12 and 14 forming the outer side of the tattoo apparatus, a driving motor 16 installed inside the upper part case 12, a cam 18 which is provided with a cam face 18a having a tilt angle and connected to the driving shaft 16a of the driving motor 16 to form a rotatory motion portion, a longitudinal bar 20 which is installed inside the lower part case 14, and one end of which contacts to the cam face 18a on a place eccentric from the center of the end of the longitudinal bar 20 to form a longitudinal motion portion which makes a rectilinear reciprocation motion based on the rotation of the cam 18, and a needle 24 for making a scar on the skin of a body, the needle 24 being connected to another end of the longitudinal bar 20 and an elastic spring 22 so that said one end of the longitudinal bar 20 could adhere closely to the cam face 18a.

The conventional tattoo apparatus 10 of the above-described structure has the cam 18 rotated by the rotation of the driving motor 16. Then, the longitudinal bar 20 comes to perform a rectilinearly reciprocation motion by contacting to the high and low parts of the cam face 18a repeatedly. Accordingly, the needle 24 goes in and out of the exit 14a at the bottom of the lower part case 14. Here, the elastic spring 22 moves the longitudinal bar 20 to the upper part so that the needle 24 should go into the inside of the exit 14a, when the cam face 18a rotates from the high side to the low side based on the rotation of the cam 18. When the cam face 18a rotates from the low side to the high side, the longitudinal bar 20 moves to the lower part so that the needle 24 should get out of the exit 14a.

However, since the cam rotates with its cam face closely adhering to one end of the longitudinal bar, the conventional tattoo apparatus causes vibration seriously as the cam rotates at a high speed, thus making the tattooing work harder and more painful.

In addition, due to the elastic spring, the conventional tattoo apparatus requires as much force as to overcome the restitution force of the elastic spring in order to make the longitudinal bar perform a rectilinear reciprocation motion. To obtain the force, bigger driving motor has to be used and this makes the weight and volume of the tattoo apparatus heavier and larger.

FIG. 2A is a cross-sectional view illustrating another conventional tattoo apparatus, and FIG. 2B is a cross-sectional view showing the operation of the tattoo apparatus shown in FIG. 2A. As shown in FIGS. 2A and 2B, the tattoo apparatus 30 includes: upper and lower part cases 32 and 34 for forming the outer side of the tattoo apparatus, a driving motor 36 installed inside the upper part case 32, a driving shaft 38 of the driving motor 36 which is provided with a cam face 38a having a tilt angle, a rotatory bar 40 which is formed in the inside of the lower part case 34 with a hinge 40a and rotated by a cam face 34 based on the rotation of the driving shaft 38, a rectilinear bar 42, on top of which a connection end 42a is formed protruded and connected to one end of a rotatory bar 40 with a hinge 42a, for performing a rectilinear reciprocation motion by the rotation of, a longitudinal bar 40, and a needle 44, which is connected to another end of the rectilinear bar 40, for making a scar on the skin of a body.

According to the conventional tattoo apparatus 30 of the above described structure, the driving shaft 38 is rotated by the driving motor 36, and this rotates the rotatory bar 40 which contacts the high and low sides of a cam face 38a repeatedly. Accordingly, the rectilinear bar 42 performs a rectilinear reciprocation motion of getting the needle 44 in and out of an exit 34a positioned at the bottom of the lower part case 34.

The second conventional tattoo apparatus is provided with a separate rotatory bar for making the rectilinear bar perform a rectilinear reciprocation motion. So, the rotation of the rotatory bar and the rectilinear reciprocation motion of rectilinear bar are performed simultaneously, which causes much vibration and makes the tattooing work troublesome. Besides, as the tattoo apparatus is used, the connection end of the rectilinear bar becomes weaker and, at the end, it may be broken. Due to the weak connection between the needle and the rectilinear bar, the needle may be sprung out during the tattoo making and this is quite dangerous.

### SUMMARY OF THE INVENTION

It is, therefore, an object of the present invention to provide a tattoo apparatus that can reduce the vibration generated during the operation of the tattoo apparatus and improve the efficiency in making tattoo by making a rectilinear bar perform a rectilinear reciprocation motion based on the rotation of a rotatory gear and a longitudinal gear, which form a helical bevel structure together, to thereby get a needle in and out of the tattoo apparatus.

It is another object of the present invention to provide a tattoo apparatus that, can be used more conveniently by simplifying the rectilinear reciprocation structure of the rectilinear bar to thereby reduce the weight and volume of the tattoo apparatus.

Further, it is another object of the present invention to provide a tattoo apparatus that can control the exposed length of the needle with the grip of the tattoo apparatus to thereby control the depth of scar made on the skin of a body and express diverse tattoos.

In accordance with an aspect of the present invention, there is provided an apparatus for making tattoo, including: a case for forming the outer side of the tattoo making apparatus to have a space inside; a driving motor installed inside the upper part of the case; a rotatory gear connected to a driving shaft of the driving motor to thereby form a rotatory motion portion; a longitudinal gear tooth-engaged with the rotatory gear to thereby form a longitudinal motion portion; an eccentric shaft, which is installed eccentrically from the center of the longitudinal gear rotatably, for converting rotatory motion to an up and down rectilinear motion; a rotatory bar, of which one end is connected to the eccentric shaft rotatably, for performing an up and down rectilinear reciprocation motion based on the rotatory motion of the eccentric shaft; a rectilinear bar, of which one end is connected to another end of the rotatory bar with: a hinge and another end is installed exposed out of the lower end of the case, for performing an up and down rectilinear reciprocation motion based on the rotation of the rotatory bar; a rectilinear bar anti-rolling unit for preventing the rectilinear bar from rolling right and left; a needle, of which one end is connected to another end of the rectilinear bar, for making a scar on the skin of a body; a connection unit for connecting the needle to another end of the rectilinear bar; and a grip which is connected to the lower part of the case to form a gripping portion, and provided with an exit for guiding the needle in the lower part of the case.

In accordance with another aspect of the present invention, the rectilinear bar anti-rolling unit of the tattoo apparatus includes: a rectilinear member having plane guiding faces on both sides of the upper end of the rectilinear bar; and a rectilinear bar guiding member having a penetration hole for penetrating the rectilinear bar from up end to down end, and a guiding member for guiding the rectilinear member from up to down with the guiding faces contacting the rectilinear member, the both sides of the guiding member being formed apart from each other as long as the distance between the guiding faces of the rectilinear member at both sides of the upper end.

In accordance with another aspect of the present invention, it is desirable to form the up-down length of the guiding member of the rectilinear bar guiding member longer than the up-down moving range of the rectilinear bar so that the guiding faces of the rectilinear member should not get out of the up-down length of the guiding member.

In accordance with another aspect of the present invention, the connection unit for connecting the needle to another end of the rectilinear member includes: an elastic supporting member which is provided with a connection groove at another end of the rectilinear bar and divided into a plurality of parts of a predetermined length in the longitudinal direction to elastically support the needle inserted and connected to the connection groove; a first male screw part formed on the external circumferential surface of the elastic supporting member; and a needle fixing nut, which is provided with a first female screw part corresponding to the male screw portion on the internal circumferential surface of the needle fixing nut, for fixing the needle inserted and connected to the connection groove by being engaged with the male screw portion of the elastic supporting member.

The elastic supporting member can be divided into three or four parts.

In accordance with another aspect of the present invention, the male screw portion of the elastic supporting member that forms the lower end of the rectilinear bar may be formed in a double structure of up and down parts corresponding to each other, and the up and down parts of the male screw portion are connected to each other with the needle fixing nut. The rotatory gear and the longitudinal gear may be designed in a helical bevel gear structure.

In accordance with another aspect of the present invention, the tattoo apparatus further includes: a rotatory bar motion range limiting member, which is formed at one end of the eccentric shaft in front of the longitudinal gear with the rotatory bar connected to the eccentric shaft at the center, for limiting the range of one end of the rotatory bar moving right and left.

In accordance with another aspect of the present invention, the tattoo apparatus may further include: a packing member for maintaining the connection state of the grip at a proper area of the external circumferential surface in the lower part of the case.

In accordance with another aspect of the present invention, the tattoo apparatus further includes: a unit for controlling the exposed length of the needle, which is provided with a second male screw part formed on the external circumferential surface in the lower part of the case and a second female screw part formed at a proper area of the internal circumferential surface of the grip.

In accordance with another aspect of the present invention, the exit of the grip is formed in such a diameter that a predetermined amount of ink for making tattoo could be maintained by the surface tension of the ink, and when the needle goes in and out of the exit, the ink could be stuck to the end of the needle and injected to the scar on the skin.

### BRIEF DESCRIPTION OF THE DRAWINGS

The above and other objects and features of the present invention will become apparent from the following description of the preferred embodiments given in conjunction with the accompanying drawings, in which:

FIG. 1A is a cross-sectional view showing a conventional tattoo apparatus;

FIG. 1B is a cross-sectional view describing the operation of the tattoo apparatus shown in FIG. 1A;

FIG. 2A is a cross-sectional view illustrating another conventional tattoo apparatus;

FIG. 2B is a cross-sectional view showing the operation of the tattoo apparatus shown in FIG. 2A;

FIG. 3 is an exploded view illustrating a tattoo apparatus in accordance with an embodiment of the present invention;

FIG. 4 is a perspective view showing the tattoo apparatus in accordance with the embodiment of the present invention;

FIG. 5A is a partial perspective view describing the connection of a rectilinear bar and a rectilinear bar guiding member for preventing the rectilinear bar from rolling in accordance with the embodiment of the present invention;

FIG. 5B is a partial perspective view illustrating the operation of the rectilinear bar shown in FIG. 5A;

FIG. 6A is a cross-sectional view showing the tattoo apparatus of FIG. 4, which is cut out along a line A-A;

FIG. 6B is a cross-sectional view describing the operation of the tattoo apparatus shown in FIG. 6A;

FIG. 7A is a cross-sectional view showing the tattoo apparatus of FIG. 4, which is cut out along a line B-B;

FIG. 7B is a cross-sectional view describing the operation of the tattoo apparatus shown in FIG. 7A; and

FIG. 8 is a perspective view showing a rectilinear bar in accordance with another embodiment of the present invention.

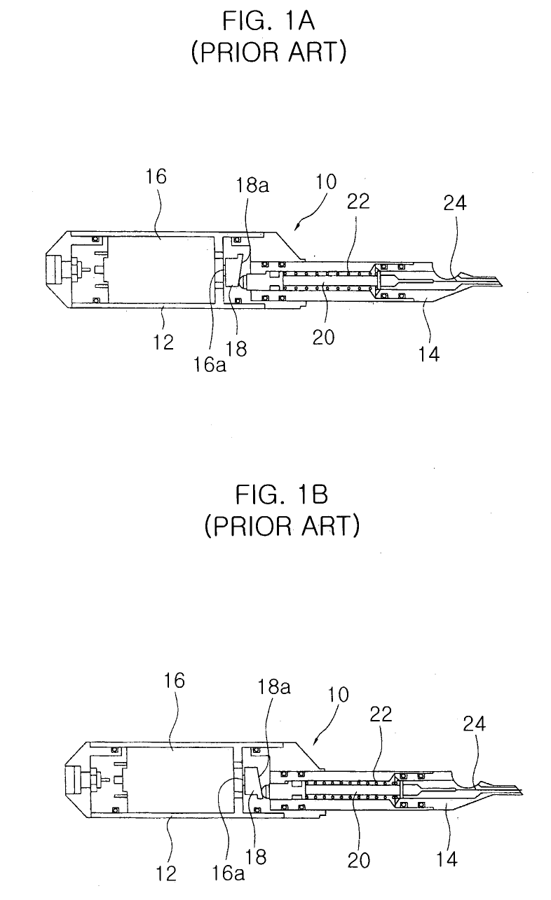

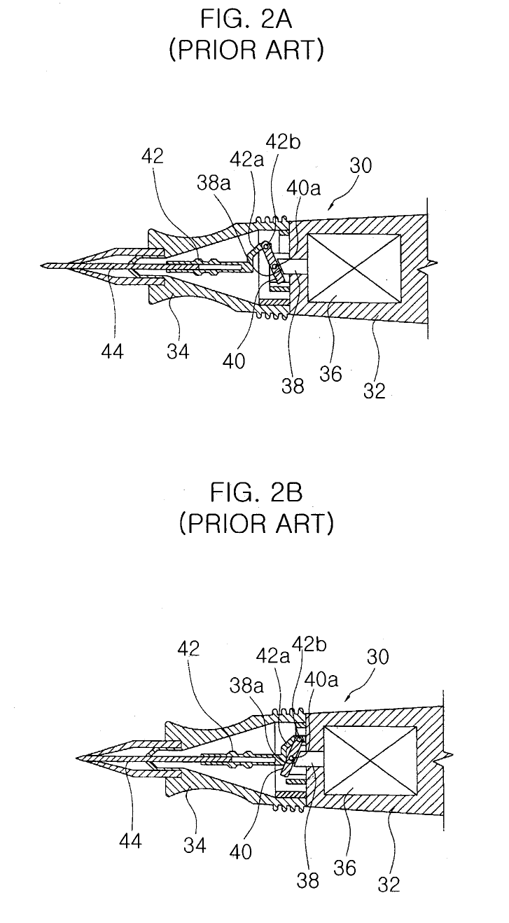

### DETAILED DESCRIPTION OF THE INVENTION

Other objects and aspects of the invention will become apparent from the following description of the embodiments with reference to the accompanying drawings, which is set forth hereinafter.

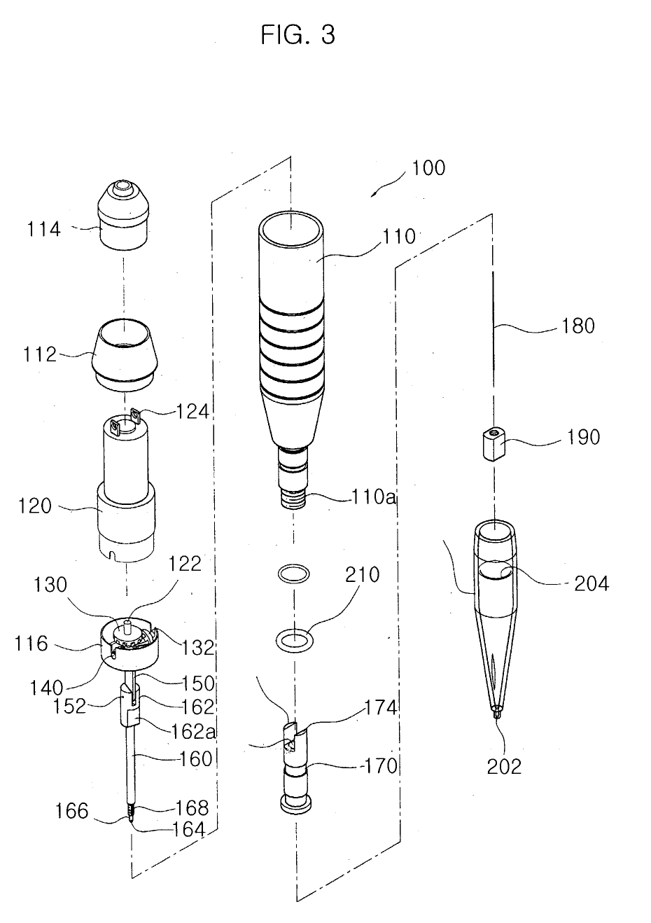

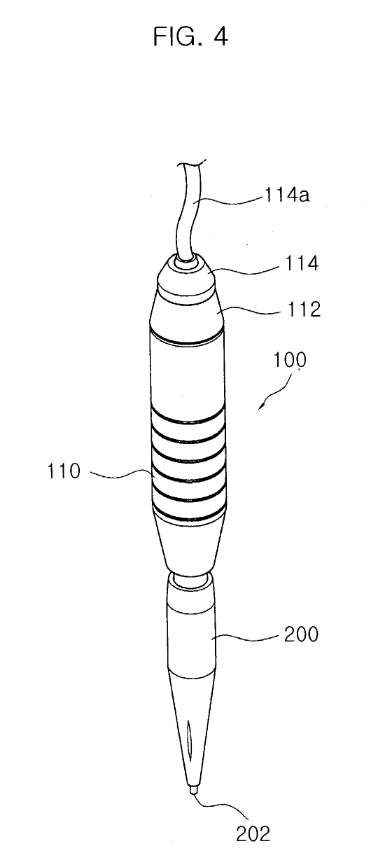

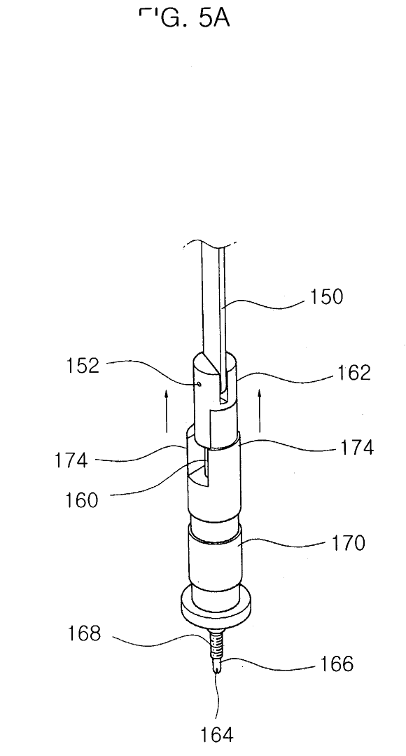

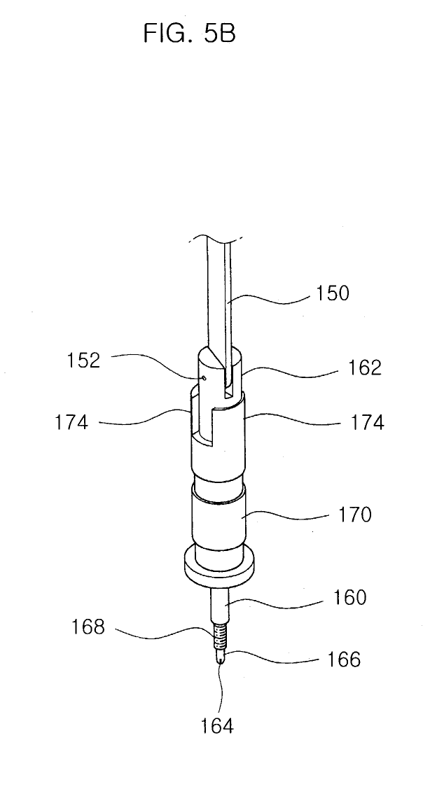

FIG. 3 is an exploded view illustrating a tattoo apparatus in accordance with an embodiment of the present invention, and FIG. 4 is a perspective view showing the tattoo apparatus in accordance with the embodiment of the present invention. FIG. 5A is a partial perspective view describing the connection of a rectilinear bar and, a rectilinear bar guiding member for preventing the rectilinear bar from rolling in accordance with the embodiment of the present invention, and FIG. 5B is a partial perspective view illustrating the operation of the rectilinear bar shown in FIG. 5A.

As shown in FIGS. 3 to 5, the tattoo apparatus 100 of the present invention includes: a case 110 for forming the outer side of the tattoo apparatus to have a space inside; a driving motor 120 installed inside the case 110; a rotatory gear 130 connected to a driving shaft 122 of the driving motor 120; a longitudinal gear 132 tooth-engaged with the rotatory gear 130; an eccentric shaft 140 installed eccentrically from the center of the longitudinal gear 132; a rotatory bar 150 for converting the rotatory motion into a rectilinear motion; a rectilinear bar 160, of which one end is connected to another end of the rotatory bar 150 with a hinge 152, for performing rectilinear reciprocation motion from up and down; a rectilinear bar anti-rolling unit for preventing the rectilinear bar 160 from rolling right and left; a needle 180, of which one end is connected to another end of the rectilinear bar 160, for making a scar on the skin of a body; a needle fixing unit for connecting the needle 180 to another end of the rectilinear bar 160; and a grip 200 which is connected to the lower part of the case 110 to form a grip portion, and provided with an exit 202 for guiding the needle 180.

The tattoo apparatus 100 described above is operated as following. First, the driving motor 120 is operated upon the supply of power. The rotation of the driving motor 120 also rotates the rotatory gear 130 which is connected to the driving shaft 122 of the driving motor 120. Successively, the longitudinal gear 132, which is tooth-connected to the rotatory gear 130, is rotated, too.

In the mean time, the eccentric shaft 140 installed eccentrically from the center of the longitudinal gear 132 is rotated by the rotation of the longitudinal gear 132. Then, this rotatory motion of the eccentric shaft 140 is converted into a rectilinear reciprocation motion by the rotatory bar 150.

As described above, the rotatory bar 150 converts the rotatory motion of the eccentric shaft 140 to a rectilinear reciprocation motion. So, the rectilinear bar 160 performs rectilinear reciprocation motion up and down. This motion makes the needle 180 get in and out of the exit 202 of the grip 200 to thereby make a scar on the skin. Here, little amount of ink for tattoo is maintained inside the exit 202 of the grip 200 by its surface tension at the tip of the needle 180 and injected into the scar when the needle 180 makes a scar on the skin.

To describe the tattoo apparatus of the present invention more in detail, first, there is a case 110 which forms the outer side of the tattoo apparatus 100. The case 110 has a space inside and it perforates up and down of the case. Here, the inner space has a wide upper part and relatively narrow lower part.

In the upper part of the case 110 described above, a motor fixing case 112 is formed to fix the driving motor 120 to be described later, and an electric wire fixing case 114 is formed in the upper part of the motor fixing case 112 to connect an electric wire 114a to an access terminal 124 of the driving motor 120, electrically.

The driving motor 120 drives the gears 130 and 132 so that the needle 180 could move up and down by the rectilinear reciprocation motion of the rectilinear bar 160. The driving motor 120 is set up in the inner space of the case 110.

In the upper part of the driving motor 120, the access terminal 124 is formed electrically connected to the electric wire 114a, and at the center of the lower part of the driving motor 120, the driving shaft 122 is formed connected to the rotatory gear 130, to be described later on.

The rotatory gear 130 is used to convey the driving force generated by the operation of the driving motor 120. The rotatory gear 130 is connected orthogonal to the driving shaft 122 in the lower part of the driving motor 120.

The longitudinal gear 132 is used to convey the rotatory force generated by the rotation of the rotatory gear 130 to the rotatory bar 150, which will be described later on. The longitudinal gear 132 is tooth-connected to the rotatory gear 130 at a right angle. That is, the connection of the rotatory gear 130 and longitudinal gear 132 forms a helical bevel structure.

The longitudinal gear 132, which forms a helical bevel structure with the rotatory gear 130, is installed in the lower part of the driving motor 120 rotatably by the eccentric shaft 140 in the central part of the longitudinal gear 132. To be more specific, the longitudinal gear 132 is connected to the lower part of the driving motor 120, being supported by one side of the eccentric shaft 140 so that it can be rotated by an eccentric shaft supporting member 116 which supports the eccentric shaft 140 to be rotatable.

The eccentric shaft 140 is used to convert the rotatory motion of the longitudinal gear 132 to an up and down rectilinear motion. The eccentric shaft 140 is formed in the lower part of the driving motor 120 and supported by the eccentric shaft supporting member 116, which supports both sides of the eccentric shaft 140 rotatably.

The rotatory bar 150 performs up and down reciprocation motion based to the rotatory motion of the eccentric shaft 140. The upper part of the rotatory bar 150 is connected to the eccentric shaft 140 rotatably, and the lower part of the rotatory bar 150 is connected to the upper part of the rectilinear bar 160, which will be described later on, with a hinge 152. The rotatory bar 150 of the above described structure performs up and down reciprocation motion by the rotation of the eccentric shaft 140 to thereby make the rectilinear bar 160 perform rectilinear reciprocation motion.

The rotatory motion of the rotatory gear 130 and longitudinal gear 132, which forms a helical bevel structure, is caused by the operation of the driving motor 120 and converted to an up and down reciprocation motion by the eccentric shaft 140 and rotatory bar 150.

The rectilinear bar 160, which performs a rectilinear reciprocation motion up and down by the rectilinear reciprocation of the rotatory bar 150, moves the needle 180 up and down. The upper part of the rectilinear bar 160 is connected to the lower part of the rotatory bar 150 with a hinge 152.

Meanwhile, the lower part of the rectilinear bar 160 is connected with the lower part of the rotatory bar 150 with the hinge 152. Here, the lower part of the rectilinear bar 160 is exposed out of the lower part of the case 110 through the exit.

The rectilinear bar anti-rolling unit is used to minimize the right and left rolling of the rectilinear bar 160, which performs an up and down rectilinear motion. It includes a rectilinear member 162 having plane guiding faces 162a on both sides of the upper part of the rectilinear bar 160, and a rectilinear bar guiding member 170 provided with a guiding member 174 for guiding the rectilinear member 162 up and down with the guiding faces 162a contacting the rectilinear bar 160. Here, the rectilinear bar guiding member 170 has a penetration hole 172 for the rectilinear bar 160 penetrating through, and the both sides of the upper part of the guiding member 174 are formed apart from each other as much as the distance between the guiding faces 162a at both sides of the rectilinear member 162.

In the above described structure, the lengths of the guiding member 174 of the rectilinear bar guiding member 170 and the guiding faces 162a of the rectilinear member 162 are formed longer than the up-down range of the rectilinear bar 160 so that the guiding faces 162a of the rectilinear member 162 should not get out of the length of the guiding member 174. Here, the up-down length of the guiding faces 162a of the rectilinear member 162 is formed shorter than that of the guiding member 174 of the rectilinear bar guiding member 170.

As described above, the rectilinear bar guiding member 170 of the rectilinear bar anti-rolling unit is forced to be inserted to the inner space through the open lower part of the case 110 and then connected. In the inside of the case 110, the rectilinear member 162 of the rectilinear bar 160 is placed between the guiding members 174 of the rectilinear bar guiding member 170. Here, the plane guiding faces 162a at both sides of the rectilinear member 162 contact the inner faces of the guiding member 174 of the rectilinear bar guiding member 170.

Meanwhile, to place the guiding faces 162a of the rectilinear member 162 between the guiding members 174 of the rectilinear bar guiding member 170, the rectilinear bar 160 should be penetrated through the penetration hole 172 of the rectilinear bar guiding member 170 which is fixed inside the lower part of the case 110 so that both guiding faces 162a of the rectilinear member 162 could be inserted between both sides of the guiding member 174 of the rectilinear bar guiding member 170. Here, the lower part of the rectilinear member 162 is stuck in the upper part of the guiding member 174, and the both guiding faces 162a of the rectilinear member 162 are inserted between the guiding member 174 of the rectilinear bar guiding member 170 by rotating the rectilinear bar 160 to the right and left.

In this state where the elements of the tattoo apparatus are formed in the above described structure, when the driving motor 120 is operated, the guiding faces 162a of the rectilinear member 162 become to perform a rectilinear reciprocation motion up and down without getting out of the up-down range of the guiding member 174 of the rectilinear bar guiding member 170. Therefore, the right and left rolling of the rectilinear bar 160 is minimized.

The needle 180 is an instrument that makes a scar on the skin of a body. One end of the needle 180 is connected to and supported by the lower part of the rectilinear bar 160 with a connection unit for connecting the needle 180 to the lower part of the rectilinear bar 160.

The connection unit for connecting the needle 180 to another end of the rectilinear bar 160 includes: an elastic supporting member 166 which is provided with a connection groove 164 in the lower part of the rectilinear bar 160 and divided into three or four parts of a predetermined length in the longitudinal direction to elastically support the needle 180 inserted and connected to the connection groove 164; a male screw portion 168 formed on the external circumferential surface of the elastic supporting member 166; and a needle fixing nut 190, which is provided with a female screw portion 192 corresponding to the male screw portion 168 on the internal circumferential surface of the needle fixing nut 190, for fixing the needle 180 inserted and connected to the connection groove 164 by being engaged with the male screw portion 168 of the elastic supporting member 166.

In the connection unit for connecting the needle 180 to another end of the rectilinear bar 160 described above, the needle fixing nut 190 fixes the needle 180, elastically supported by the elastic supporting member 166 of the rectilinear bar 160, to the lower part of the rectilinear bar 160 by being engaged with the male screw portion 168 in the lower part of the rectilinear bar 160 exposed under the lower part of the case 110.

The grip 200 is a portion of the tattoo apparatus 100 manually held by a tattooist, when tattoo is made. It has an exit 202 for guiding the needle 180 in and out of the tattoo apparatus at the bottom of the grip 200.

The grip 200 of the above described structure makes the tattooist hold the tattoo apparatus 100 and protects the needle 180 as well as protecting the tattooist from the needle 180.

The exit 202 of the grip 200 is formed in such a diameter that a predetermined amount of ink 220 for tattoo could be maintained by its own surface tension and when the needle 180 goes in and out of the tattoo apparatus, the ink 220 could be stuck to the tip of the needle 180 and injected to the scar on the skin.

That is, the tattoo apparatus 100 of the present invention performs tattoo making with a predetermined amount of ink 220 for tattoo at the end of the exit 202 of the grip 200.

The upper end of the grip 200 having the above described structure is connected to the lower part of the case 110. Here, the case 110 includes a packing member 210 for maintaining the connection with the grip 200 at a proper area of the external circumferential surface in the lower part of the case 110.

Also, provided to the tattoo apparatus 10 of the present invention is a control unit for controlling the exposed length of the needle 180. The needle control unit includes a male screw part 110a formed on the external circumferential surface in the lower part of the case 110 and a female screw part 204 formed at a proper area of the internal circumferential surface of the grip 200.

The control unit for controlling the exposed length of the needle 180 controls the length of the needle 180, exposed to the outside of the exit 202 of the case 200, by moving the grip 200 up and down by rotating it in the locking or unlocking direction.

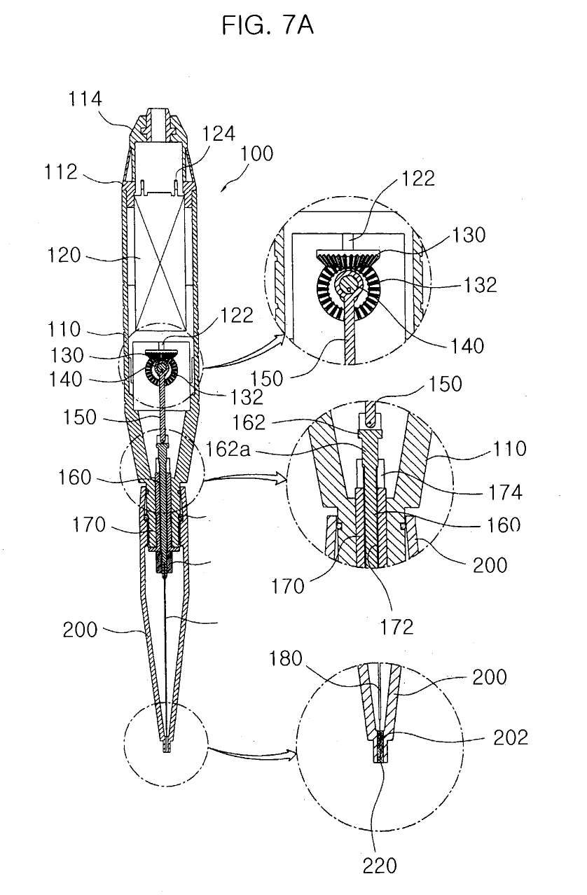

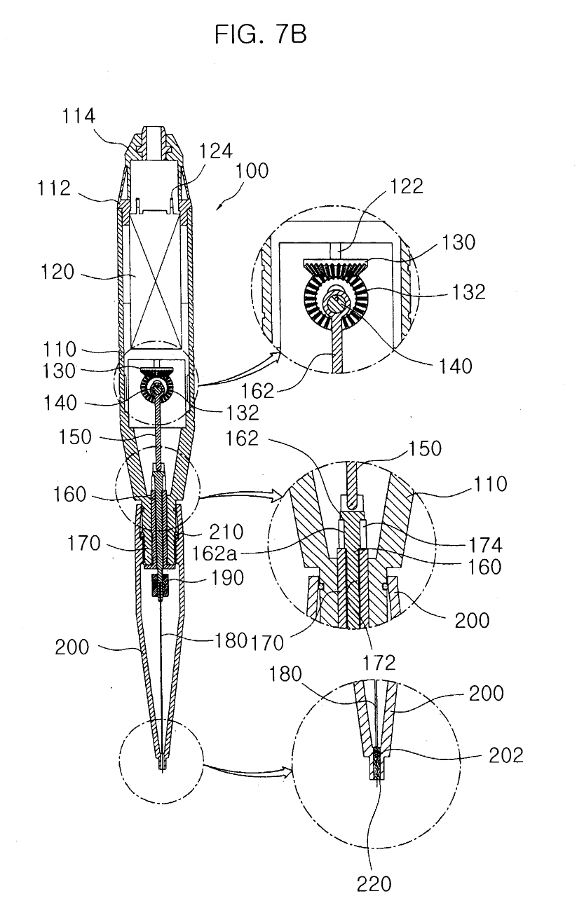

The tattoo apparatus of the present invention can further include a rotatory bar motion range limiting member 142 (see FIG. 7), which is provided at one end of the eccentric shaft 140 in front of the longitudinal gear 132 with the rotatory bar 150 connected to the eccentric shaft 140 at the center, for limiting the range of one end of the rotatory bar 150 moving right and left.

The rotatory bar motion range limiting member 142 limits the motion range of the rotatory bar 150 so that one end of the rotatory bar 150, which moves right and left around the eccentric shaft 140, should not get out of a predetermined range.

The elements of the tattoo apparatus 100 is assembled by connecting the rotatory bar 150 and the rectilinear bar 160 with a hinge 152, connecting the upper end of the rotatory bar 150 to the eccentric shaft 140 rotatably, and connecting one end of the eccentric shaft 140 to the longitudinal gear 132. The above structure is connected to the lower part of the driving motor 120 rotatably and supported by the eccentric shaft supporting member 116. Here, the longitudinal gear 132 is tooth-engaged with the rotatory gear 130 connected to the driving shaft 122 of the driving motor 120 in a helical bevel structure. The assembly of the driving motor 120, rotatory gear 130, longitudinal gear 132, eccentric shaft 140, rotatory bar 150 and rectilinear bar 160 is inserted into the inner space of the case 110 in the upper part. Here, the rectilinear member 162 of the rectilinear bar 160 penetrates the penetration hole 172 of the rectilinear bar guiding member 170, which is forced to be inserted into the inner space of the case 110 and gets fixed through the lower part of the case 110, and the lower end of the rectilinear member 162 becomes stuck in the upper end of the guiding member 174.

In this state, when the driving motor 120 is rotated right and left, the guiding faces 162a at both sides of the rectilinear member 162 are inserted between both sides of the guiding member 174 in the upper end of the rectilinear bar guiding material 170. As shown above, when the guiding faces 162a at both sides of the rectilinear member 162 are inserted between both sides of the guiding member 174 in the upper end of the rectilinear bar guiding material 170 through the right and left rotation of the driving motor 120, the lower end of the rectilinear bar 160 is exposed outside of the lower end of the case 110.

When the lower end of the rectilinear bar 160 is exposed outside of the case 110, the upper end of the needle 180 is connected to the connection groove 164 in the lower end of the rectilinear bar 160 to have the elastic supporting member 166 support the structure preliminarily. Then, the needle fixing nut 190 is engaged with the male screw portion 168 formed on the external circumferential surface of the elastic supporting member 166 to fix the needle 180 to the lower end of the rectilinear bar 160.

Subsequently, a motor fixing case 112 is connected to the upper part of the case 110 to fix the driving motor 120 in the inner space of the case 110. The upper part of the motor fixing case 112 is connected to an electric wire fixing case 114 electrically connected with the access terminal 124 of the driving motor 120 through electric wire 114a.

Meanwhile, the grip is connected to the lower end of the case 110. Here, the grip 200 is connected to a proper area of the external circumferential surface in the lower part of the case 110, and the connection state is maintained preliminarily by the packing member 210. In this state, when the grip 200 is rotated in the locking direction; the male screw part 110a formed on the external circumferential surface in the lower end of the case 110 and the female screw part 204 formed in a proper area on the internal circumferential surface of the grip 200 are combined, which is a secondary connection.

The tattoo apparatus 100 of the present invention is assembled as described above. Here, the grip 200 can be moved up or down to thereby control the length of the needle 180 exposed outside the exit 202 of the grip 200, whose female screw part 204 on the internal circumferential surface is engaged with the male screw part 110a on the external circumferential surface of the case 110, by rotating the grip 20 in the locking or unlocking direction.

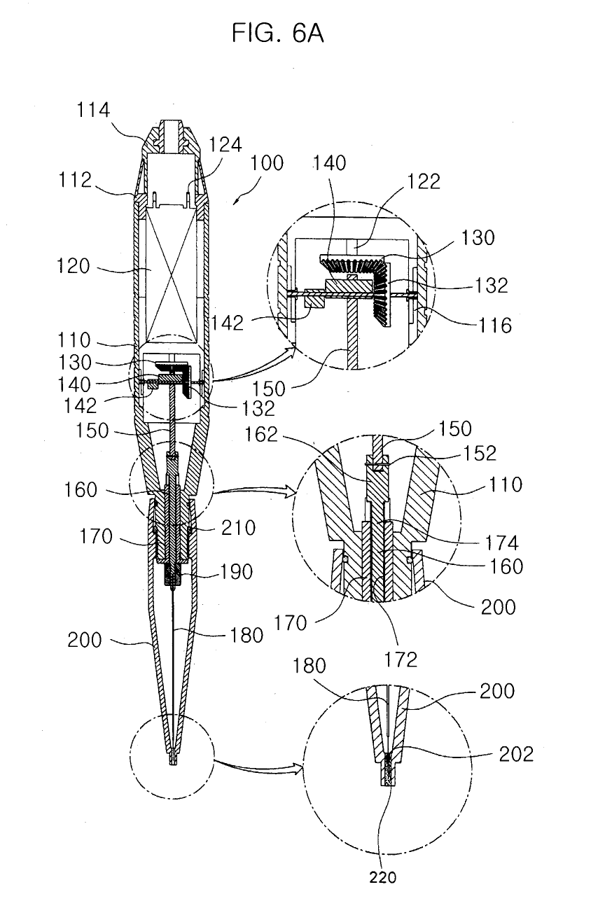

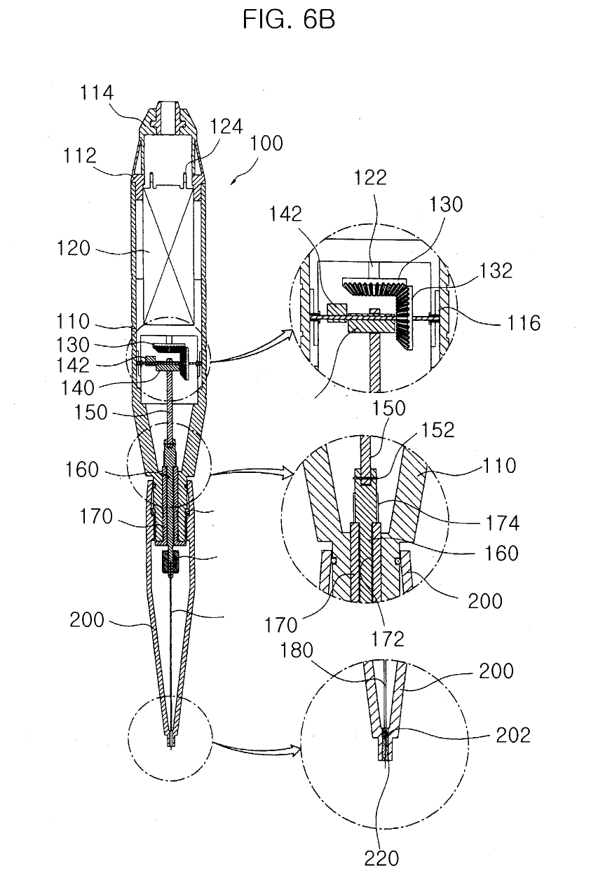

FIG. 6A is a cross-sectional view showing the tattoo apparatus of FIG. 4, which is cut out along a line A-A; and FIG. 6B is a cross-sectional view describing the operation of the tattoo apparatus shown in FIG. 6A. FIG. 7A is a cross-sectional view showing the tattoo apparatus of FIG. 4, which is cut out along a line B-B; and FIG. 7B is a cross-sectional view describing the operation of the tattoo apparatus shown in FIG. 7A.

As shown in FIGS. 6A, 6B, 7A and 7B, the tattoo apparatus 100 of the present invention is operated by maintaining a predetermined amount of ink 220 inside the exit 202 of the grip 200 by using surface tension of the ink 220, putting the line end of the exit 202 of the grip 200 closely on the skin of a body to be tattooed, and supplying power and operating the diving motor 120.

When power is supplied and thus the diving motor 120 is operated, the rotatory gear 130 connected to the driving shaft 122 of the driving motor 120 is rotated, and the rotation of the rotatory gear 130 is led to the operation of the longitudinal gear 132 which is tooth-connected to the rotatory gear 130.

Due to the operation of the longitudinal gear 132, the eccentric shaft 140 installed eccentrically from the center of the longitudinal gear 132 rotatably is rotated. The rotatory motion of the eccentric shaft 140 is converted into a rectilinear reciprocation motion by the rotatory bar 150.

Since the rotatory bar 150 converts the rotatory motion of the eccentric shaft 140 into a rectilinear reciprocation motion, the rectilinear bar 160 performs a rectilinear reciprocation motion up and down. Then, the needle 180 goes in and out of the exit 202 of the grip 200, making a scar on the skin.

While the tattoo apparatus 100 is operated as described above, the guiding faces 162a of the rectilinear member 162 performs an up and down rectilinear reciprocation motion without getting out of the up-down range of the guiding member 174 of the rectilinear bar guiding member 170.

If the ink 220 for tattoo held inside the exit 202 of the grip 200 is all used up in the middle of tattoo making, the exit 202 of the grip 200 is dipped into the ink to thereby have a little amount of ink 220 for tattoo maintained in the inside of the exit 202 of the grip 200 with its own surface tension, and then the tattoo making is resumed.

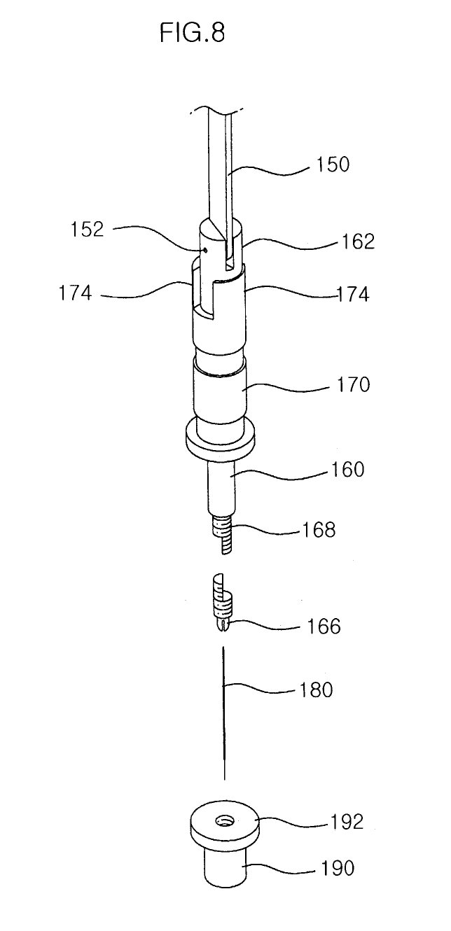

FIG. 8 is a perspective view showing a rectilinear bar in accordance with another embodiment of the present invention. The structure of the rectilinear bar 160 of FIG. 8 is for abandoning the blood-stained elements, which is used once, and replacing it with new ones. As illustrated in the drawing, the male screw portion 168 of the elastic supporting member 166, which forms the lower end of the rectilinear bar 160 has a double structure severed into up and down parts in correspondence to each other so that they are connected to each other with a fixing nut 190.

To be more in detail, a portion supporting the needle 180 in the lower end of the male screw portion 168 of the elastic supporting member 166 which forms the lower end of the rectilinear bar 160 is separated from the rectilinear bar 160 so that the it could be attached to or detached from the lower end of the rectilinear bar 160 by locking or unlocking the fixing nut 190. Therefore, after the tattoo apparatus 100 is used once, the fixing nut 190 is disconnected to detach and get rid of the portion (A) that supports the needle 180 in the lower end of the male screw portion 168 from the lower end of the rectilinear bar 160. Here, the blood-stained needle 180, fixing nut 190 and grip 200 are disposed, along with the portion (A) supporting the needle 180 in the lower end of the male screw portion 168. Then, new needle 180, the portion (A) supporting the needle 180 in the lower end of the male screw portion 168 fixing nut 190 and grip 200 are replaced.

As described above, the present invention can provide a more hygienic tattoo apparatus 100 by detaching the portion supporting the needle 180 in the lower end of the male screw portion 168 of the elastic supporting member 166, which forms the lower end of the rectilinear bar 160, from the rectilinear bar 160 after the tattoo apparatus 100 is used once.

Meanwhile, the tattoo apparatus 100 of the present invention may further include on top of the fixing nut 190 a protection member 192 for preventing blood from being inserted into the inside of the lower end of the rectilinear bar guiding member 170. The protection member 192 is formed in a diameter corresponding to the external diameter of the lower end of the rectilinear bar guiding member 170.

Due to this protection member 192 formed on the upper end of the fixing nut 190, blood spattered in during tattoo making is blocked from the lower part of the rectilinear bar guiding member 170 by the bottom of the protection member 192. Thus, it is possible to provide a more hygienic tattoo apparatus 100.

As described above, the tattoo apparatus 100 of the present invention can reduce the level of violation and enhance the efficiency in tattoo making by rotating the rotatory gear and longitudinal gear, which are formed in a helical bevel gear structure, to make the rectilinear bar perform a rectilinear reciprocation motion and thereby getting the needle in and out of the tattoo apparatus.

In addition, the tattoo apparatus 100 of the present invention can be used more conveniently by simplifying the rectilinear reciprocation structure of the rectilinear bar and making the weight and volume of the tattoo apparatus 100 lighter.

Since the length of the needle exposed outside can be controlled with the grip and thus the depth of scar made on the skin of a body can be controlled, more diverse tattoo can be expressed.

While the present invention has been described with respect to certain preferred embodiments, it will be apparent to those skilled in the art that various changes and modifications may be made without departing from the scope of the invention as defined in the following claims.

What is claimed is:

1. An apparatus for making tattoo, comprising:
a case for forming the outer side of the tattoo making apparatus to have a space inside;
a driving motor installed inside the upper part of the case;
a rotatory gear connected to a driving shaft of the driving motor to thereby form a rotatory motion portion;
a longitudinal gear tooth-engaged with the rotatory gear to thereby form a longitudinal motion portion;
an eccentric shaft, which is installed eccentrically from the center of the longitudinal gear rotatably, for converting rotatory motion to an up and down rectilinear motion;
a rotatory bar, of which one end is connected to the eccentric shaft rotatably, for performing an up and down rectilinear reciprocation motion based on the rotatory motion of the eccentric shaft;
a rectilinear bar, of which one end is connected to another end of the rotatory bar with a hinge and another end is installed exposed out of the lower end of the case, for performing an up and down rectilinear reciprocation motion based on the rotation of the rotatory bar;
a rectilinear bar anti-rolling means for preventing the rectilinear bar from rolling right and left;
a needle, of which one end is connected to another end of the rectilinear bar, for making a scar on the skin of a body;
a connection means for connecting the needle to another end of the rectilinear bar; and
a grip which is connected to the lower part of the case to form a gripping portion, and provided with an exit for guiding the needle in the lower part of the case.

2. The apparatus as recited in claim 1, wherein the rectilinear bar anti-rolling means includes:
a rectilinear member having plane guiding faces on both sides of the upper end of the rectilinear bar; and
a rectilinear bar guiding member having a penetration hole for penetrating the rectilinear, bar from up end to down end, and a guiding member for guiding the rectilinear member from up to down with the guiding faces contacting the rectilinear member, the both sides of the guiding member being formed apart from each other as long as the distance between the guiding faces of the rectilinear member at both sides of the upper end.

3. The apparatus as recited in claim 2, wherein the up-down length of the guiding member of the rectilinear bar guiding member is formed longer than the up-down moving range of the rectilinear bar so that the guiding faces of the rectilinear member should not get out of the up-down length of the guiding member.

4. The apparatus as recited in claim 1, wherein the connection means for connecting the needle to another end of the rectilinear member includes:
an elastic supporting, member which is provided with a connection groove at another end of the rectilinear bar and divided into a plurality of parts of a predetermined length in the longitudinal direction to elastically support the needle inserted and connected to the connection groove;
a first male screw part formed on the external circumferential surface of the elastic supporting member; and
a needle fixing nut, which is provided with a first female screw part corresponding to the male screw portion on the internal circumferential surface of the needle fixing nut, for fixing the needle inserted and connected to the connection groove by being engaged with the male screw portion of the elastic supporting member.

5. The apparatus as recited in claim 4, wherein the male screw portion of the elastic supporting member that forms the lower end of the rectilinear bar is formed in a double structure of up and down parts corresponding to each other, and the up and down parts of the male screw portion are connected to each other with the needle fixing nut.

6. The apparatus as recited in claim 1, further comprising:
a rotatory bar motion range limiting member, which is formed at one end of the eccentric shaft in front of the longitudinal gear with the rotatory bar connected to the eccentric shaft at the center, for limiting the range of one end of the rotatory bar moving right and left.

7. The apparatus as recited in claim 1, further comprising:
a packing member for maintaining the connection state of the grip at a proper area of the external circumferential surface in the lower part of the case.

8. The apparatus as recited in claim 1, further comprising:
a means for controlling the exposed length of the needle, which is provided with a second male screw part formed on the external circumferential surface in the lower part of the case and a second female screw part formed at an area of the internal circumferential surface of the grip.

9. The apparatus as recited in claim 1, wherein the exit of the grip is formed in such a diameter that a predetermined amount of ink for making tattoo could be maintained by the surface tension of the ink, and when the needle goes in and out of the exit, the ink could be stuck to the end of the needle and injected to the scar on the skin.

10. The apparatus as recited in claim 1, wherein the rotatory gear and the longitudinal gear are formed in a structure of helical bevel gear.
